(19) United States
(12) Patent Application Publication
(10) Pub. No.: US 2007/0106182 A1
(43) Pub. Date: May 10, 2007

(54) Method for determining and measuring frontal head posture and frontal view thereof

(76) Inventor: G. William Arnett, Santa Barbara, CA (US)

Correspondence Address: CHRISTIE, PARKER & HALE, LLP, PO BOX 7068, PASADENA, CA, 91109-7068, US

(21) Appl. No.: 11/252,411

(22) Filed: Oct. 17, 2005

(51) Int. Cl. A61B 5/103

(52) U.S. Cl. 600/587

(57) ABSTRACT

A method for determining and measuring profile and frontal head posture and front views thereof to establish a 3-D model of a patient's head and face. In the method, a patient places his or her head in a natural head position and establishes a postural sagittal plane. Additionally, the practitioner may move the patients head to a corrected sagittal plane. If still necessary, an anatomic sagittal plane that comprises an anatomical midline of the patient's face when viewed from a frontal view is constructed. An profile anatomical coronal plane is located and aligned with the frontal anatomical sagittal plane. Next, an anatomical transverse plane is established through the soft tissue nasion and aligning it perpendicularly to the anatomical sagittal and coronal planes. By developing the 3 planes a 3-D reference frame is established from which soft tissue and hard tissue landmarks are identified and measured.

### FIELD OF THE INVENTION

The invention relates generally to the field of facial measurement and analysis, and more particularly to a system and method for determining the frontal head posture and measuring the frontal view of the face and underlying hard tissue structures, e.g., such as dentoskeletal structures, and combining such analysis with profile analysis and treatment planning to develop a system and method for full 3D facial analysis.

### BACKGROUND

The study of facial aesthetics is old. Facial attractiveness, while innately recognizable by lay people and artists, has in the past been difficult to quantify. Philosophers and artists have struggled for centuries to identify the concrete structural relationships that create an aesthetically pleasing face. Relationships and proportions for pleasing facial contours are well understood, but only in general terms.

It is understood that facial attractiveness can be defined by at least two main facial characteristics, namely quality of facial parts and position of facial parts. The quality of the eyes, skin, the hair, and lips alter the perception of what is beautiful. Cosmetology and the fashion industry (and to a certain extent medicine by dermatology and chemical skin peels to improve the color, tone, smoothness of the skin) are founded upon improving the quality of these features.

However, the position, shape and size of the facial features arguably have an even greater impact on facial aesthetics. For example, the position, shape and size of the cheekbones, orbital rims, nose, jaws and chin are even more major determinants of facial aesthetics, and surgeons, orthodontists, and dentists are able to affect changes to these facial features using various treatment methods.

Advancements in orthodontics and orthagnathic and craniofacial surgery have led to tremendous advancements in the available treatment for persons with congenital defects, developmental defects, victims of injury and those who simply wish to improve their facial aesthetics. It is known that changes to dentoskeletal structures, such as the teeth, cartilage and bones that underlie soft tissue will greatly influence the outward appearance of the face and head. Accordingly, for example, in orthodontics, the movement of the teeth and jaw line are important in optimizing the frontal and side profiles. While some orthodontists and orthagnathic surgeons are experienced and skillful enough to estimate what changes to the underlying hard structures will result in the most aesthetically pleasing results, for most of these professionals, standards, guidelines and specific directives are greatly preferable and result in more consistently excellent results.

The inventor has in the past developed methods of soft tissue cephalometric analysis for diagnosis and methods for cephalometric treatment planning for aesthetic correction of facial imbalance in a patient with regards to the side profile, both for undifferentiated males and females, and for differentiated male and female groups. See U.S. Pat. Nos. 5,951,498 and 6,200,278, respectively. The disclosures in U.S. Pat. Nos. 5,951,498 and 6,200,278 are incorporated herein in their entirety.

While a good profile is an essential part of good facial aesthetics, it is possible to have a good profile yet still have an overall unbalanced face if the front view has imperfections.

In the past, what has been missing is a method for frontal facial analysis that can be used to ensure that the frontal view of a patient's face is aesthetically optimized. Moreover, by incorporating a method for frontal facial analysis and treatment planning with a method for profile analysis and treatment planning, a person's entire facial balance can be optimized from all views (3D), resulting in an aesthetically pleasing facial appearance from any angle.

There accordingly remains a need for a method for frontal facial analysis that can be used to ensure that the frontal view of a patient's face is aesthetically optimized by a method of combined frontal and profile analysis and treatment planning.

### SUMMARY OF THE INVENTION

One object of the invention is to provide a method of analysis that focuses on soft tissue landmarks as opposed primarily to hard tissue to achieve consistently excellent outcomes in dental, orthodontic and surgical correction of facial imbalance in frontal view and in frontal plus profile view so that the face and head may be studied in 3D from a variety of angles.

Another object of the invention is to provide health care professionals with a discrete treatment plan to guide in precisely what action must be taken to improve facial balance whether in a frontal view, or frontal and profile views and any other views thereof.

The diagnosis and treatment normal values of the current invention will be carried out by establishing normal size, distance, and angle ranges of various facial landmarks of aesthetically pleasing faces from a population group, and having a patient place his or her head in a natural head position, and correcting that position if necessary, and establishing an anatomical position of the head through which the three perpendicular anatomical planes, namely, the sagittal, transverse and coronal planes will be correlated, and measuring soft and hard tissue landmarks to these anatomical planes to define skeletal and/or soft tissue measurements for the face, and then making orthodontic, dental, and surgical corrections to improve the facial balance and aesthetics as necessary.

### BRIEF DESCRIPTION OF THE DRAWINGS

FIG. 1 is a diagrammatic front perspective view showing a human head and the perpendicular sagittal SP, coronal CP and transverse TP or the coplanar anatomical planes ASP, ACP, ATP.

FIG. 2 is a diagrammatic frontal facial view showing the sagittal plane SP or anatomical sagittal plane ASP passing through the midline of the face and landmarks on the face and facial outline landmarks.

FIG. 3 is a diagrammatic frontal facial view showing the sagittal plane SP or anatomical sagittal plane ASP, the transverse plane TP or anatomical transverse plane through Na′ ATP-Na′ and various measurement landmarks and lines for the middle and lower ⅓rds of the face measured perpendicular to ASP and parallel to ATP-Na′.

FIG. 4 is a diagrammatic frontal facial view showing the sagittal plane SP or anatomical sagittal plane ASP and the transverse plane TP or anatomical transverse plane passing through nasion ATP-Na′, with levels drawn though the eyes, upper canine teeth, lower canine teeth, inferior border of the lower jaw, and chin, with the cheekbone contour also illustrated. Also shown are the intercanthal distance ICD and the alar base width ABW.

FIG. 5 is a diagrammatic right side facial profile view showing the position of the coronal plane CP or anatomical coronal plane drawn through subnasale ACP-Sn and facial profile landmarks.

FIG. 6 is a diagrammatic frontal facial view showing width measures of the facial outlines.

FIG. 7 is diagrammatic frontal facial view showing facial symmetry measurements.

### DETAILED DESCRIPTION OF THE INVENTION

In the invention, the frontal head posture and measurements will be used in conjunction with the profile postural head position and measurements in order to create a 3-D model of the face and head.

A frontal and profile head position images will be taken with conventional CT scans, cone beam CT, 3D photography, 2D photography, scattered light photography, laser scanning, profile and frontal cephalometrics, ultrasound and magnetic resonance imaging, or any other imaging technique that presently exists or in the future may be developed.

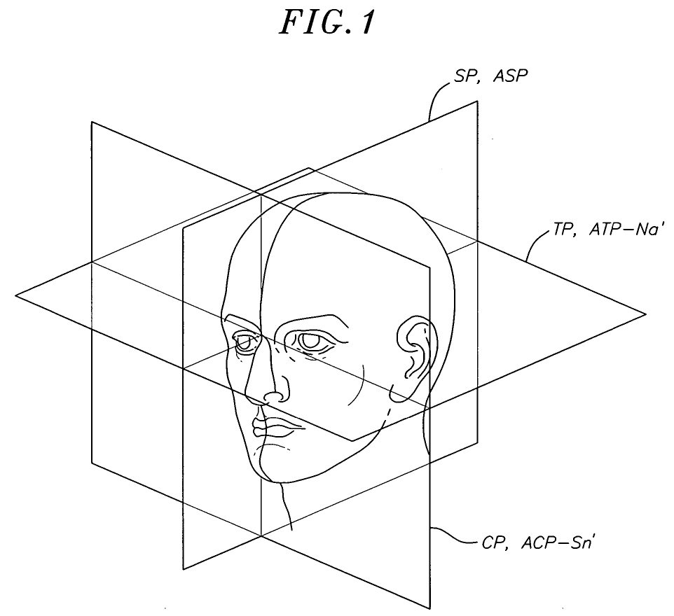

In order to position the head in the proper anatomical position, it is necessary to properly orient the head with respect to three perpendicular anatomical reference planes, namely the sagittal plane SP, the transverse plane TP, and coronal plane CP. These three anatomical planes of references are shown in FIG. 1 with reference to a diagrammatic head and face.

The sagittal plane SP is perpendicular to the floor, runs front to back and up and down. The anatomical sagittal plane is generally utilized to measure the width of the face. The anatomical sagittal plane ASP is a vertical plane that passes through the centerline of the patient's face from front to back, top to bottom, and divides it symmetrically. If the head is properly positioned and balanced, the sagittal plane will pass longitudinally through the centerline of the nose. If a patient tilts his/her chin up or down along the sagittal plane, this will not affect the distances of landmarks to the sagittal plane. However, if the head is tilted to the left or right shoulder, or the head is turned left or right, then the landmarks will move out of alignment with the sagittal plane.

The transverse (or axial) plane TP is a plane parallel to the ground and goes from left to right, front to back. The anatomical transverse plane is generally used to measure the height of the face on the left and right sides. By way of example, when a facially balanced and symmetrical head is in a perfect transverse head position, symmetrical horizontal landmarks on the face and head (e.g., the pupils of the eyes) will lie on the same transverse plane. Turning of the head left to right will not cause the horizontal landmarks on the face and head to be taken out of the transverse plane. In contrast, if the head is tilted to the left or right shoulder, this will affect the distances of the landmarks to the transverse plane TP. If the chin is brought up or down, perpendicularly to the transverse plane TP, this will also affect the distances of the symmetrical landmarks to the transverse plane TP, but equally to both sides.

The coronal plane CP is the vertical plane of reference that is parallel to the front of the patient's face (up and down) and follows from left to right. The coronal plane (CP) is used to measure the projection of the left and right sides, and midline of the face. If the patient's face is facially balanced and properly oriented, the same landmark on the right and left side of the face will be equal distance from the coronal plan CP (see FIG. 5). Regardless of whether the face is facially balanced, tilting of the head from side to side will not affect the distances of landmarks to the coronal plane. However, if the head is turned axially from side to side or the chin is brought up or down, this will affect the distances of the landmarks to the coronal plane.

Since the face is ideally laterally symmetrical viewed from the frontal view, the distances of symmetrically located anatomical features, e.g., the two pupils of eye, from the sagittal plane and from the transverse plane should be the same in a symmetrical and balanced face.

Thus, it can be appreciated that the proper position of the head in the three planes of reference is important in order to create the proper 3-D diagnosis and treatment plan of the head. In order to position the head in the correct orientation, the following exemplary technique may be used. This exemplary technique is representative of the possible techniques that can be used and is not meant to be limiting. Proper posturing is imperative to insure the reliability of any analysis and treatment based on the captured image.

There are three steps to head orientation during imaging and analysis. First, the patient will be asked to look straight ahead into a mirror at his or her pupils and establish a natural head position. In order to do this, the patient will look into a mirror and level his/her head by moving it up and down (bringing the chin up and down) along the sagittal plane. The patient will also turn his or her head from left side to right side along the transverse plane and then look straight into the mirror into his or her own pupils. The patient will also tilt his head from shoulder to shoulder while looking into a mirror to align his head relative to the coronal plane. Carrying out these movements while the patient looks directly into the pupils orients the patient's head into a natural head position.

The natural or postural head position thus established is based on the perspective of the patient looking in a mirror into his or her own eyes. During the process it is important the patients joints are seated, the teeth are at fist contact, and the lips are passively related. The postural head position will thus be oriented in 3-D space relative to the perpendicular sagittal, transverse and coronal planes. The straight ahead vertical and horizontal planes thus established by the patient from his or her own perspective are referred to herein as the “postural sagittal plane PSP”, the “postural transverse plane PTP” and the “postural coronal plane PCP”. However, the postural sagittal plane, the postural transverse plane and the postural coronal plane may or may not in fact be anatomically accurate and could be out of alignment with one or more of the anatomical sagittal, transverse and coronal planes. The postural head position may be altered by postural habits which the patient has acquired or imaging techniques and equipment may make postural head position impossible for the patient to achieve during image procurement.

The second step is to determine the corrected postural position. To make any required corrections, for example, if the practitioner notices that the head is tilted incorrectly relative to one or more of the three anatomical planes, the practitioner can either ask the patient to move his/her head as necessary, or the practitioner can physically move the patients head for him/her. The patient's determined head orientation, including positions in the transverse plane TP, coronal plane CP and sagittal plane SP, may be altered by the practitioner, if deemed necessary, for example as described below, to a corrected transverse plan CTP, corrected coronal plane CCP and a corrected sagittal plane CSP.

Step three is determining the anatomical head position. As used herein, the term “anatomical sagittal plane” ASP is the true (anatomical) midline of the face when viewed from the frontal view. The ASP is used to correct the patient's frontal postural head position after the initial imaging and self-effected positioning conducted by the patient and practitioner adjustments as described above.

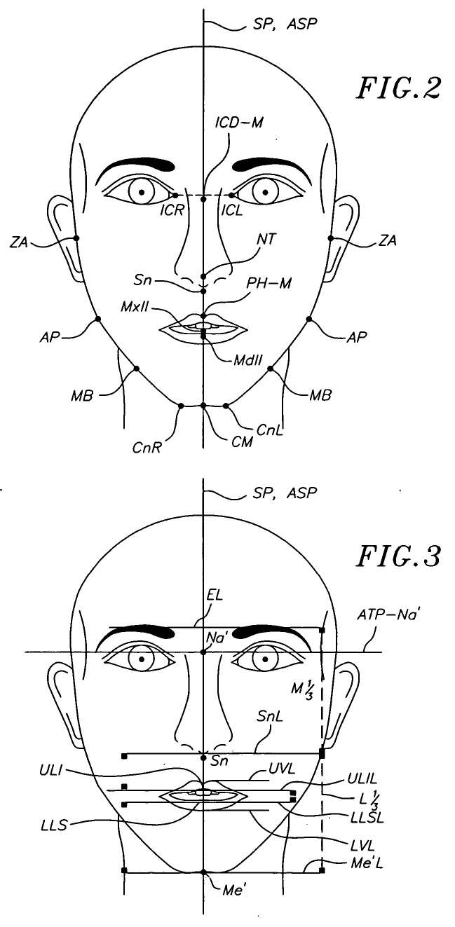

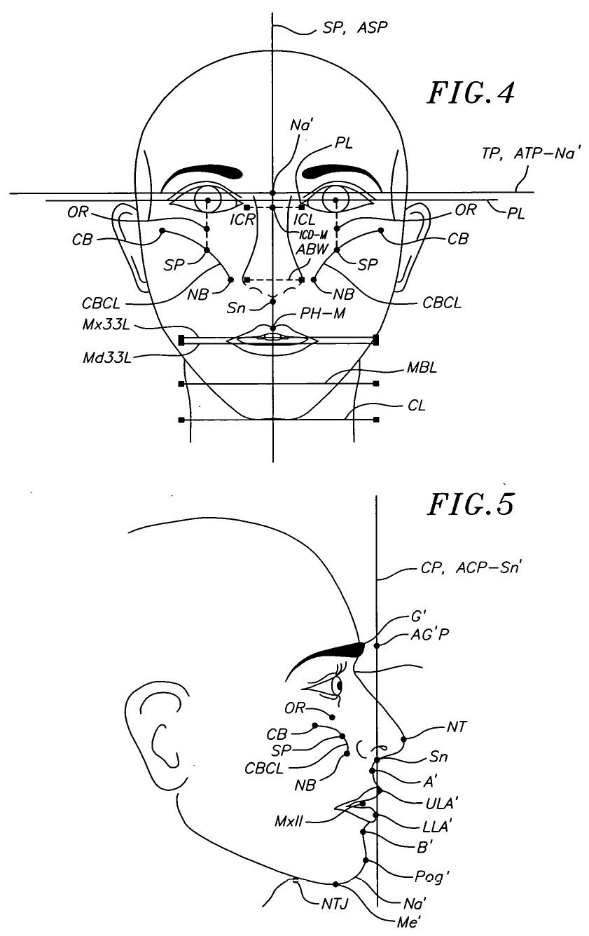

As shown in FIGS. 2 and 4, the coordinates of the corrected, anatomical sagittal plane ASP passes through the midpoint of the intercanthal distance ICD-M and the midpoint of the upper lip as defined by the midline of the philtrum PH-M, with the ASP (ICD-M to PH-M) extending above the hair line to below the soft tissue menton Me′. The anatomical sagittal plane ASP is perpendicular to the floor and is used to measure hard and soft tissue structures of the face as described below. Of course, if the postural sagittal plane passes through the same points as what would be the anatomical sagittal plane, then no correction is required. Inasmuch as the above identified coordinates (ICD-M to PH-M) are used to position the landmarks of the anatomic sagittal plane ASP (which will correspond to the postural sagittal vertical plane in a face that is correctly postured by the patient and/or practitioner in the mirror and is naturally correctly balanced), it is possible to entirely skip over the steps the patient undertakes for self-evaluation of the postural or sagittal plane and go directly to the steps undertaken by the medical professional to determine the anatomical sagittal plane ASP.

The patient starts the process in the mirror. This is verified or corrected by the practitioner as necessary and the anatomic sagittal plane ASP can then be applied after imaging is procured. (This process is done this way because some patients are not capable of displaying natural head position because of head orientation habits and imaging equipment and techniques may make it difficult if not impossible for the patient to assume the natural head position.)

Tilting of the head to the left or right shoulder is corrected by constructing the anatomical sagittal plane (ASP). Rotation of the sagittal plane about the axis of the head to the left or right is corrected by equalizing the distance from the left and right pupils to the sagittal plane.

Likewise, as shown in FIG. 5, the anatomical coronal plane ACP can be derived. The plane is constructed through subnasale Sn and a point 6-12 mm in front of soft tissue glabella G′ (anterior glabella point AG′P). The ACP is perpendicular to the floor, perpendicular to the anatomical sagittal plane ASP and is the anatomical correction for profile postural head position. The exact position of anterior glabella point AG′P is determined by clinical examination of the patient while looking in straight ahead gaze into the mirror without interference by image acquisition equipment. Head position correction by the doctor or operator (corrected postural position) may be necessary to establish the position of anterior glabella point AG′P location. When midface retrusion is diagnosed, the subnasale point is moved 1-3 mm anterior. Midface retrusion is defined by a long nose, deficient alar base, poor incisor upper lip support, upright upper lip and/or thick upper lip.

After orientation of the head via the anatomic sagittal plane and the anatomic coronal plane the transverse plane is established as a perpendicular to the other two planes.

The three anatomical planes (if correction is required), are used in several ways. Herein below, the term “anatomical planes” will be sometimes used to refer to the postural, doctor or operator corrected position or to the anatomical planes. First, the anatomical planes are used to measure the width equality of the left and right sides of the face at a variety of levels (i.e., zygomatic arches or angles of the mandible, etc.) Second, the anatomical planes can be used to measure the distance to the same landmarks (i.e., pupils of the eyes, angles of the mandible, upper and lower canine teeth) on both the left and right sides of the face to determine if these landmarks are on the same level to the transverse plane and/or are perpendicular to the anatomical sagittal plane, or in other words, used to make a measurement of vertical symmetry. Third, the anatomical planes can be used to orient the face for measurement of the height and width and proportionality of those distances. Fourth, the anatomical planes can be used to orient the face for vertical measurement of the face (i.e., upper ⅓ height, upper incisor exposure, etc.) A fifth use of the anatomical planes is to orient the face for a variety of structural measurements (i.e., alar base width [soft tissue], maxillary intercanine width [hard tissue], etc.) As a sixth use, the anatomical planes can be used to orient the face for shape and contour measurements (i.e., cheekbone contour, bridge of nose line).

Key landmarks of the face and head are measured to the anatomical planes. These include some or all of the following:

a. Any hard or soft tissue structures selected by the examining medical professional or other operator. These can include, for example, soft tissue or skeletal angles of the mandible.

b. Vertical structures, for example middle ⅓ of face, upper and lower lip heights.

c. outline structures, i.e., zygomatic arch width, mandibular body width.

d. Structure levels, i.e., eyes, maxillary canines, chin.

e. Midlines structures, i.e., tip of nose, upper incisor midline, and chin.

f. Left and right sides of face, i.e., cheekbone height of contour, angles of mandible.

g. Shape and contour symmetry, i.e., base of nose width, cheekbone contour.

The anatomical planes are also used to orient the face for measurement of any internal structures, such as the base of nose, bony piriform rim, zygomatic buttresses etc.) There are innumerable possibilities which can be measured. Specific measurements would be chosen by the practitioner depending upon specialty.

To facilitate the above measurements, frontal soft tissue and hard tissue facial analysis landmarks are identified using the postural, doctor or operator corrected, or anatomical planes. FIGS. 2-7 refer to some sample measurements which can be used for diagnosis and treatment planning in the area of orthodontics and orthagnathic procedures. A craniofacial surgeon may wish to use different and/or additional landmarks to measure based upon his needs. A table of measurements can be utilized and additional measurements can be taken depending upon the practitioners specific needs.

Referring again to FIG. 2, there is shown a diagrammatic frontal facial view with the sagittal plane SP and the anatomical sagittal plane ASP, midline measurement structures, outline structures, and right and left structures shown. These midline landmarks include the inner canthal distance midline ICD-M, nasal tip NT, philtrum midline PH-M, the midline between the two central maxillary incisors Mx11, the midline between the two central mandible incisors Md11, and the chin midline CM. The anatomical sagittal plane is drawn through ICD-M and PH-M. Outline landmarks include left and right chin Cn, angle points AP, mandibular body points MB, and zygomatic arches ZA.

FIG. 3 is a diagrammatic frontal facial view with vertical landmarks and measurements being shown. The measurements are measured parallel to the ATP-Nalline. All measurements are taken with the head in the anatomical aligned position relative to the sagittal, transverse and coronal planes. Additional landmarks and horizontal one-third lines are shown, namely a horizontal line EL at the eyebrow level and a horizontal line SnL at subnasale level which define the middle third (M⅓), and the horizontal line SnL at the subnasale level and a horizontal line Me′L through soft tissue menton point Me′ which defines the lower third (L⅓). Additional horizontal lines are shown, namely a horizontal line ULIL through upper lip inferior point (ULI), a horizontal line LLSL through the point lower lip superior (LLS), a horizontal line UVL at the junction of the upper vermillion and skin, a horizontal line LVL at the junction of the lower lip vermillion and the skin. The interlabial gap is defined by the gap between the horizontal line ULIL and the horizontal line LLSL. The upper lip length is SnL to ULIL and the lower lip length is LLIL to Me′L. The upper vermillion height is UVL to ULIL while the lower lip vermillion is LLIL to LVL.

FIG. 4 also shows additional landmarks and various measurements. The sagittal plane (SP) and it co-planar anatomical sagittal (ASP), are depicted. All measurements are taken with the head in the anatomical aligned position relative to the sagittal, transverse and coronal planes. In FIG. 4, levels are depicted for the pupils of the eye PL, maxillary canines Mx33L, mandibular canines Md33L, body of the mandible MBL, and bottom of the chin CL. Additionally, the cheekbone contour, intercanthal distance ICD, and alar base width ABW are depicted. “Level” is when structures are on a line that is perpendicular to the anatomical sagittal plane ASP. For example, the pupils are level when they both are on a line perpendicular to the anatomical sagittal plane. Mirrored structures, which are otherwise supposed to be symmetrical located, are canted when they do not fall on a line perpendicular to the ASP. (i.e., maxillary canine on left down by 2 mm relative to the right canine).

In FIG. 4 it should be noted that the pupil line PL often is not perpendicular to the anatomical sagittal plane ASP. Thus, the pupil line PL should not be used as a base line to level other facial structures to (i.e., Mx33 level). Cheekbone contour lines CBCL can be drawn through the cheekbone CB, subpupil SP and nasal base NB landmarks on the left and right sides of the face. When these lines are flat, a cheekbone augmentation is indicated.

As an additional method to measure vertical symmetry and facial heights the anatomical transverse plane (ATP) is drawn through soft tissue nasion Na′ ATP-Na′ and is drawn perpendicular to the anatomical coronal plane ACP and the anatomic sagittal plane ASP. The transverse plane TP and anatomical transverse plane through nasion (ATP-Na′) are depicted in FIG. 4. The anatomical transverse plane through Na′ ATP-Na′ is used as a reference plane to measure the vertical position of hard and soft tissue structures. Measurements from the anatomical transverse plane through Na′ ATP-Na′ to the same landmark on the left and right side of the face indicate vertical symmetry of facial structures (i.e., maxillary molar mesial buccal cusp tips, orbital rims, left and right chin). If the practitioner desires, the anatomical transverse plane (ATP) can be drawn at any level and does not have to be through soft tissue Na′.

FIG. 5 is a diagrammatic right side facial profile view depicting profile measurements measured concurrently to the frontal examination, with the anatomical coronal plane ACP shown passing through subnasale Sn. All measurements are taken with the head in the anatomical aligned position relative to the sagittal, transverse and coronal planes. The anatomical coronal plane ACP can be determined as set forth in U.S. Pat. Nos. 5,951,498 (for an undifferentiated group of males and females) and U.S. Pat. No. 6,200,278 (for differentiated groups of males and females) for determining the true vertical line TVL described therein, with the anatomical sagittal plane ASP passing through the true vertical line TVL as described therein. The anatomical transverse plane (ATP) is by definition perpendicular to the anatomical transverse and sagittal planes and the TVL described therein. The contents of U.S. Pat. Nos. 5,951,498 and 6,200,278 are incorporated herein by reference. Other points on the facial profile, such as the soft tissue glabella G′, anterior glabella point AG′P, nasal tip NT, soft tissue A point A′, upper lip anterior ULA′, maxillary incisor Mx11, lower lip anterior LLA′, soft tissue B point B′, soft tissue pogonion Pog′, soft tissue menton Me′ and neck throat junction NTJ are shown. The cheekbone contour line CBCL on the right side of the face is shown drawn through the cheekbone CB, subpupil SP and nasal base NB landmarks. Together, measurements from the anatomical sagittal plane ASP and the anatomical coronal plane ACP will allow mapping of these points in 3-D space. In the method of the invention, a left side facial profile view should be taken, as the left and right sides of the faces are often not perfectly symmetrical, and horizontal distances from the landmarks, e.g., the cheekbone CB, subpupil SP and nasal base NB landmarks on the left and right sides of the face can differ as measured to the ACP.

When desired or necessary an appropriate facial landmark identification object or mark can be applied to the skin prior to imaging. The type of identification landmark may be different depending on the type of imaging being used (i.e., cone beam CT, 2-D or 3-D photography, scattered light photography, laser scanning, ultrasound, magnetic resonance imaging and cephalometric x-ray). For example, objects as simple as pen markings or small (e.g., round) adhesive markers which do not distort the imaging modality being used (i.e., metal distorts CT imaging) can be used.

The anatomical sagittal plane ASP, the anatomical coronal plane ACP, and the anatomical transverse plane ATP, as defined above, are perpendicular. By perpendicularly aligning the anatomical sagittal plane ASP (ICD-M to PH-M), and the anatomical coronal plane ACP through subnasale (ACP-Sn), as well as the anatomical transverse plane ATP through Nasion (ATP-Na′) will establish a 3-D reference frame from which all landmarks (both soft tissue and hard tissue) can be identified and measured, which in turn are used to mathematically measure key features of the face and head. These figures will allow an accurate 3D plot of facial landmarks and features to be determined, and therefore will guide health care professionals in treatment planning and execution of facial improvement-procedures, such as orthodontics, orthagnathic and craniofacial surgery. The assignment of the planes to specific landmarks such as the anatomical transverse plane at nasion ATP-Na′ may be altered at the practitioner's discretion if deemed desirable.

To determine the head position relative to the coronal plane (CP) taken from a profile view, patients are assessed clinically according to a modified version of Arnett and Bergman', in natural head position, uppermost condyle, and with passive lips. See Arnett G W and Bergman R T. Facial keys to orthodontic diagnosis and treatment planning. Part I. Am J Orthod Dentofac Orthop 1993; 103(4):299-312; and Arnett G W and Bergman R T. Facial keys to orthodontic diagnosis and treatment planning. Part II. Am J Orthod Dentofac Orthop 1993; 103(5):395-411, the contents of which are incorporated herein by reference.

There are three steps to head orientation during imaging and analysis. First, the patient will be asked to look straight ahead into a mirror at his or her pupils and assumes the postural coronal position (PCP).

The patient starts the process in the mirror. This is verified or corrected by the practitioner as necessary and the anatomic coronal plane ACP (former profile TVL) can then be applied after imaging is procured. (This process is done this way because some patients are not capable of displaying natural head position because of head orientation habits and/or imaging equipment and technique prevent the patient from proper posture.)

The patient will then level his/her head by moving it up and down (bringing the chin up and down) along the sagittal plane. The patient will also turn his or her head from side to side along the transverse plane, and tilt his head from side to side (if necessary, along the coronal plan) and then look straight into the mirror into his or her own pupils. Doing this orients the frontal head posture vertically perpendicular to the floor and from rotating left or right. With the same direction, the profile is simultaneously oriented to profile postural head position. So far, these steps are all taken from the perspective of the patient looking into his or her own eyes.

The second possible step is to establish a corrected postural position. To make any required corrections, the practitioner can either ask the patient to move his/her head as necessary, or the practitioner can physically move the patients head for him/her. The patient's determined head orientation (including the postural profile true vertical line) may be altered by the practitioner, if deemed necessary, for example as described below.

Step three is to determine the anatomical head position. As used herein, the term “anatomical coronal plane” ACP is, defined as a plane which is used to correct the patients profile postural head position after the initial imaging and self-effected positioning conducted by the patient and practitioner adjustments as described above to locate the postural coronal plane or true vertical line (TVL). The anatomical coronal plane ACP is the true profile head position of the face when viewed from the profile view. The line is constructed through subnasale and a point 6-12 mm in front of soft tissue glabella (anterior glabella point). This line is perpendicular to the floor and is the anatomical correction for profile postural head position. The exact position of anterior glabella point within the range is determined by clinical examination of the patient while looking in straight ahead gaze into the mirror.

FIG. 6 is a diagrammatic frontal facial view showing some facial outlines and midlines. Selective distances are shown including the distance between the midline hair line Tr′ and menton Me′, the intercanthus distance ICD between the medial edges of the left and right eyes, and the outercanthus distance OCD between the lateral edges of the left and right eyes OCL and OCR, respectively, the alar base width ABW that extends between the alar base crease left alaL and alar base crease right alaR, chin width CnW and lastly the commissure width CmW that extends between the commissure left CmL and CmR. Also shown are the left and right zygomatic arches Zy′L and Zy′R, the gonion left and gonion right, Go′L and Go′R, respectively, and the chin left CnL and CnR.

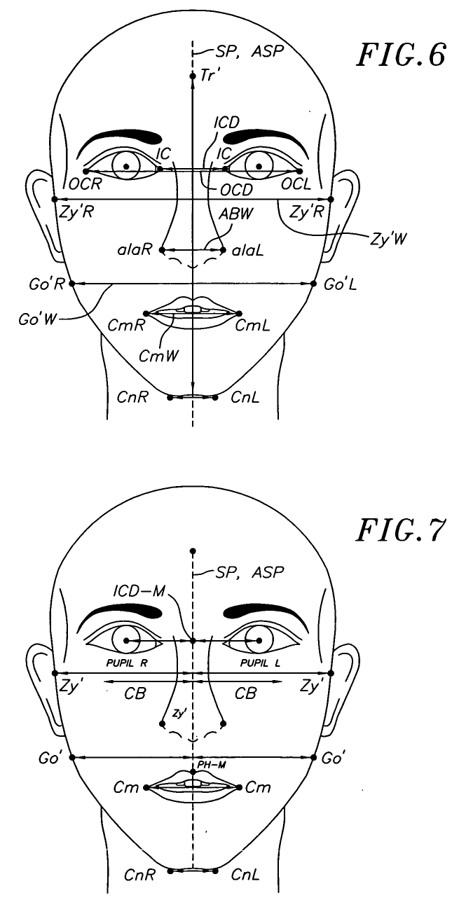

FIG. 7 is diagrammatic frontal facial view showing facial symmetry. The anatomical sagittal plane ASP passes through the inner canthal midline ICD-M and the philtrum midline PH-M. From the anatomical sagittal plane ASP, lateral distances to certain structures are measured to determine symmetry. These include, for example, the distances from the zygomatic arch left Zy′L and the zygomatic arch right Zy′R to the anatomical sagittal plane ASP, the distances from the cheek bone left CB′L and cheek bone right CB′R to the anatomical sagittal plane ASP, and distances from the gonion left Go′L and gonion right Go′R to the anatomical sagittal plane ASP.

Table 1 shows various measures from a frontal examination.

TABLE 1
PHOTO FRONTAL EXAMINATION

| MEASUREMENTS | Normal |
|---|---|
| Midlines: (frontal View) | |
| ICD-M (inner canthal distance | All on ICD-M to PH-M line |
| midline) | |
| NT (nasal tip) | |
| PH-M (philtrum midline) | |
| Mx11 (incisor midline) | |
| Md11 (incisor midline) | |
| CM (chin midline) | |
| Symmetry: (frontal view) | |
| Zy′R (zygomatic arch right) | Measured to ICD-M to PH-M line |
| Zy′L (zygomatic arch left) | Equal right and left |
| Go′R (Md angle right) | |
| Go′L (Md angle left) | |
| Me′R (chin right) | |
| Me′L (chin left) | |
| CBR (cheekbone right) | |
| CBL (cheekbone left) | |
| Levels: (frontal view) | |
| pupil L-pupil R (interpupil) | All parallel to ATP-Na′ or |
| Mx3L-Mx3R (Mx intercanine) | perpendicular to ASP |
| Md3L-Md3R (Md intercanine) | |
| CmL-CmR (intercommissure) | |
| Md3L-Md3R (intercanine) | |
| Widths: (frontal view) | |
| pupil L-pupil R (globe width) | Measured parallel to ASP-Na′ |
| ICL-ICR (inner canthal width) | or perpendicular to ASP |
| OCL-OCR (orbital width) | |
| alaL-alaR (nasal base width) | |
| CmL-CmR (mouth width) | |
| Me′L-Me′R (chin width) | |
| Outline: | |
| Tr′-Me′M (facial height) | |
| Zy′L-Zy′R (midface width) | |
| Go′L-Go′R (lower face width) | |

Table 1 is just exemplary of some of the measurements that can be taken and is not intended to be limiting in any way.

Although embodiments of the present invention have been described in detail hereinabove in connection with certain exemplary embodiments, it should be understood that the invention is not limited to the disclosed exemplary embodiments, but, on the contrary is intended to cover various modifications and/or equivalent arrangements included within the spirit and scope of the present invention, as defined in the appended claims.

What is claimed is:

1. A method for determining and measuring frontal, rotational, and profile head posture views thereof, comprising:
having a patient place his or her head in a natural head position and establishing postural coronal, sagittal, and transverse planes;
adjusting the patient's head, if necessary, to establish the coronal, sagittal, and transverse planes;
anatomically defining coronal, sagittal, and transverse planes;
and measuring soft and hard tissue landmarks to the three anatomical planes to define skeletal and soft tissue measurements of the face from a three dimensional view.

2. The method of claim 1, wherein the patient places his or her head in the natural head position and establishes the frontal postural head position by looking into a mirror at his or her pupils, leveling his or her head up and down along a sagittal plane and moving it side to side along a transverse plane in order to orient the frontal posture vertically and from left to right.

3. The method of claim 1, wherein the frontal anatomical sagittal plane that comprises an anatomical midline of the patient's face has a projection that passes through the midpoint of the intercanthal distance (ICD-M) on the bridge of the nose and the midpoint of the upper lip as defined by the center of the philtrum (PH-M) and the upper lip cupid's bow.

4. The method of claim 3, wherein the frontal anatomical sagittal plane has a projection that extends from above the patient's hairline to below the patient's soft tissue menton.

5. The method of claim 1, wherein the determined anatomical frontal, rotational, and profile head postures are used to develop the sagittal, coronal, and transverse planes to measure the patient's face.

6. The method of claim 1, wherein the determined frontal anatomical sagittal plane is used to measure the distance to same landmarks on both the left and right sides of the face to determine if these landmarks are on the same level and/or are perpendicular to the anatomical sagittal plane to make a measurement of vertical symmetry.

7. The method of claim 6, wherein the landmarks on both the left and right sides of the face are selected from the group consisting of pupils of the eyes, maxillary canine teeth, mandibular canine teeth, and inferior border of the chin.

8. The method of claim 1, wherein the determined sagittal, coronal, and transverse planes are used to orient the face for measurement of the height and width and proportionality of those distances.

9. The method of claim 1, wherein the determined sagittal, coronal, and transverse planes are used to orient the face for vertical measurement of the face.

10. The method of claim 9, wherein the vertical measurement of the face are selected from the group consisting of the upper ⅓ height, lower ⅓ height, upper lip length, lower lip length, upper incisor exposure, upper incisor crown height, interlabial gap, upper vermillion height, and lower vermillion height.

11. The method of claim 1, wherein the determined sagittal, coronal, and transverse planes are used to orient the face for structural measurements selected from the group consisting of alar base width and maxillary intercanine width.

12. The method of claim 1, wherein the determined sagittal, coronal, and transverse planes are used to orient the face for shape and contour measurements of the cheekbone and the bridge of nose line.

13. The method of claim 1, wherein key structures of the face and head are measured to the sagittal, coronal, and transverse planes are selected from at least one of the group consisting of:
a. at least one of hard and soft tissue structures;
b. vertical structures;
c. outline structures;
d. structure levels;
e. midline structures;
f. left and right sides of face;
g. shape and contour symmetry; and
h. projections of facial landmarks.

14. The method of claim 13, wherein the at least one of hard and soft tissue structures include soft tissue and skeletal angles of the mandible.

15. The method of claim 13, wherein the vertical structures are selected from the group consisting of upper ⅓ height, lower ⅓ height, upper lip length, lower lip length, upper incisor exposure, upper incisor crown height, interlabial gap, upper vermillion height, and lower vermillion height.

16. The method of claim 13, wherein the outline structures are selected from the group consisting of left and right chin, mandibular angles, zygomatic arch width and mandibular body width.

17. The method of claim 13, wherein the structure levels are selected from the group consisting of eyes, maxillary canines, mandibular canines, commissures of the mouth, inferior border of the mandible and chin.

18. The method of claim 13, wherein the midlines structures are selected from the group consisting of the nasal bridge center, the nasal tip, the philtrum, the midline between the two central mandible incisors, subnasale and the midline of the chin and the midline structures are measured to the anatomical sagittal plane as defined inner canthal midline and philtrum midline.

19. The method of claim 13, wherein the left and right sides of face are selected from the group consisting of cheekbone height of contour and angles of the mandible.

20. The method of claim 13, wherein the shape and contour symmetry are selected from the group consisting of base of nose width and cheekbone contour.

21. The method of claim 1, wherein the sagittal, coronal, and transverse planes are used to orient key the face for measurement of internal structures including bone and teeth.

22. The method of claim 21, wherein the internal structures are selected from the group consisting of the piriform aperture, zygomatic arches, lateral orbital rims, zygomatic buttresses, angles of the mandible, body of the mandible, and parasymphasis of the mandible.

23. The method of claim 13, wherein after making measurements of the key structures of the face and head, frontal soft tissue and hard tissue facial analysis landmarks are identified using the anatomic frontal true vertical line.

24. The method of claim 1, wherein facial landmark identification objects are located on the face prior to imagining the face.

25. The method of claim 24, wherein the imaging of the face takes place with at least one of the group consisting of cone beam computerized tomography, 2-D photography, 3-D photography, 3-D stereo photogrammetry, scattered light photography, laser scanning and ultrasound and magnetic resonance imaging.

26. The method of claim 1, further comprising locating an anatomical transverse plane ATP through the soft tissue nasion ATP-Na′ and aligning it perpendicularly to the anatomical sagittal and coronal planes, wherein the ATP-Na′ is used as a reference line to measure the vertical position of hard and soft tissue structures.

27. The method of claim 26, wherein the anatomical transverse plane at Nasion (ATP-Na′) can be moved to a vertical level or point at a practitioner's discretion.

28. The method of claim 26, further comprising locating a anatomic coronal plane and aligning it with the anatomical sagittal plane through subnasale, and using the anatomical transverse plane through nasion to establish a 3-D reference frame from which soft tissue and hard tissue landmarks are identified and measured, which in turn are used to measure key features of the face and head in order to accurately establish a 3-D plot of facial landmarks and features to be determined.

29. The method of claim 28, wherein the anatomical coronal plane is established by having the patient position his or her head in natural head position, uppermost condyle, and with passive lips, and placing a line through the subnasale and drawn perpendicular to the natural horizontal head position.

30. The method of claim 28, wherein the anatomic coronal plane is utilized when necessary to correct the patients determined natural head position.

31. A method for determining and measuring a profile and frontal head posture and views thereof to establish a 3-D model, comprising:
having a patient place his or her head in a natural head position and establishing a postural sagittal plane;
adjusting the patient's head as necessary if at all to establish a corrected sagittal plane that comprises an anatomical midline of the patient's face when viewed from a frontal view;
constructing an anatomic sagittal plane through nasal bridge midline and the philtrum midpoint;
locating an anatomical transverse plane through the soft tissue nasion and aligning it perpendicularly to anatomic sagittal plane; and
locating a profile anatomical coronal plane and aligning it with the anatomic sagittal plane, and using the anatomical transverse plane to establish a 3-D reference frame that is usable to measure key features of the face and head in order to accurately determine facial landmarks and features.

32. The method of claim 31, wherein the patient places his or her head in the natural head position and establishes the postural sagittal plane by looking into a mirror at his or her pupils, leveling his or her head up and down along a sagittal plane and moving it side to side along a rotational plane in order to orient the frontal posture vertically and from left to right.

33. The method of claim 31, wherein the anatomical sagittal plane that comprises an anatomical midline of the patient's face has a projection that passes through the midpoint of the intercanthal distance on the bridge of the nose and the midpoint of the upper lip as defined by the center of the philtrum and the upper lip cupid's bow, and extends from above the patient's hairline to below the patient's soft tissue menton.

34. The method of claim 31, wherein the profile anatomical coronal plane is established by having the patient position his or her head in natural head position, uppermost condyle, and with passive lips, and placing a line through the subnasale and drawn perpendicular to the natural head position horizontal.

35. The method of claim 31, wherein the anatomical coronal plane is used to correct natural head position which is inaccurate, and wherein the anatomical coronal plane is defined by a line through subnasale and anterior glabella point which is about 6 to 12 mm anterior to soft tissue glabella.

36. A method for determining and measuring frontal head posture and front views thereof, comprising:
establishing an anatomical sagittal plane that comprises an anatomical midline of the patient's face when viewed from a frontal view; and
measuring soft and hard tissue landmarks to the anatomical sagittal plane to define at least one of skeletal and soft tissue measurements of the face from a frontal view.

37. The method of claim 36, further comprising locating a anatomical transverse plane through the soft tissue nasion and aligning it perpendicularly to anatomical sagittal plane; and
locating a profile anatomical coronal plane and aligning it with the anatomical sagittal plane, and using the anatomical transverse plane to establish a 3-D reference frame that is useable to measure key features of the face and head in order to accurately determine facial landmarks and features.

38. The method of claim 33, wherein the anatomic sagittal plane that comprises an anatomical midline of the patient's face has a projection that passes through the midpoint of the intercanthal distance on the bridge of the nose and the midpoint of the upper lip as defined by the center of the philtrum and the upper lip cupid's bow, and extends from above the patient's hairline to below the patient's soft tissue menton.

39. The method of claim 33, wherein the anatomical coronal plane is established by one of being anatomically drawn by anterior glabella point and subnasale, and having the patient position his or her head in natural head position, uppermost condyle, and with passive lips, and placing a line through the subnasale and drawn perpendicular to the natural horizontal head position.
